SEQUENCE LISTING (18 sequences)

SEQ ID NO: 1 - DNA, 966 bp, Ovine
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaaaatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtgcatttttacactcctccctatgggcaatggattttccataaagaaagaaaaatcatttttctagaggtctacattcaattctgtagcatacttggagaagctgtgtttaaaaggcagtcaaaaagtattcatttttgtcaaaatttcaaaattatagcctgcctttgcaatactgcagcttttaggatgaaataa

SEQ ID NO: 2 - DNA, 195 bp, Ovine
gtgcatttttacactcctccctatgggcaatggattttccataaagaaagaaaaatcatttttctagaggtctacattcaattctgtagcatacttggagaagctgtgtttaaaaggcagtcaaaaagtattcatttttgtcaaaatttcaaaattatagcctgcctttgcaatactgcagcttttaggatgaaa

SEQ ID NO: 3 - DNA, 141 bp, Ovine
atcatttttctagaggtctacattcaattctgtagcatacttggagaagctgtgtttaaaaggcagtcaaaaagtattcatttttgtcaaaatttcaaaattatagcctgcctttgcaatactgcagcttttaggatgaaa

SEQ ID NO: 4 - DNA, 966 bp, Bovine
atgcaaaaactgcaaatctctgtttatatttacctatttatgctgattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgtttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactgactccttttttagaagtcaaggtgcattttcacactcctccctatgggcaatggatgttctatagagaaagaaaactcattttcctagaggtctacattcaattctgtagcatacttggagaagctgcattgaaaaggcagtcaaaaagtattcattttggtcaaaatttcaaaattatagcctgcctttgcaatactgcagcttttaggatgaaataa

SEQ ID NO: 5 - DNA, 195 bp, Bovine
gtgcattttcacactcctccctatgggcaatggatgttctatagagaaagaaaactcattttcctagaggtctacattcaattctgtagcatacttggagaagctgcattgaaaaggcagtcaaaaagtattcattttggtcaaaatttcaaaattatagcctgcctttgcaatactgcagcttttaggatgaaa

SEQ ID NO: 6 - DNA, 141 bp, Bovine
ctcattttcctagaggtctacattcaattctgtagcatacttggagaagctgcattgaaaaggcagtcaaaaagtattcattttggtcaaaatttcaaaattatagcctgcctttgcaatactgcagcttttaggatgaaa

SEQ ID NO: 7 - DNA, 945 bp, Belgian Blue
atgcaaaaactgcaaatctctgtttatatttacctatttatgctgattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgtttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactggtgcattttcacactcctccctatgggcaatggatgttctatagagaaagaaaactcattttcctagaggtctacattcaattctgtagcatacttggagaagctgcattgaaaaggcagtcaaaaagtattcattttggtcaaaatttcaaaattatagcctgcctttgcaatactgcagcttttaggatgaaataa

SEQ ID NO: 8 - protein, 321 aa, Ovine
MQKLQIFVYIYLFMLLVAGPVDLNENSEQKENVEKKGLCNACLWRQNNKSSRLEAIKIQILSKLRLETAPNISKDAIRQLLPKAPPLRELIDQYDVQRDDSSDGSLEDDDYHVTTETVITMPTESDLLAEVQEKPKCCFFKFSSKIQHNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPEPGEEGLNPFLEVKVHFYTPPYGQWIFHKERKIIFLEVYIQFCSILGEAVFKRQSKSIHFCQNFKIIACLCNTAAFRMK

SEQ ID NO: 9 - protein, 65 aa, Ovine
VHFYTPPYGQWIFHKERKIIFLEVYIQFCSILGEAVFKRQSKSIHFCQNFKIIACLCNTAAFRMK

SEQ ID NO: 10 - protein, 47 aa, Ovine
IIFLEVYIQFCSILGEAVFKRQSKSIHFCQNFKIIACLCNTAAFRMK

SEQ ID NO: 11 - protein, 321 aa, Bovine
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVHFHTPPYGQWMFYRERKLILLEVYIQFCSILGEAALKRQSKSIHFGQNFKIIACLCNTAAFRMK

SEQ ID NO: 12 - protein, 65 aa, Bovine
VHFHTPPYGQWMFYRERKLILLEVYIQFCSILGEAALKRQSKSIHFGQNFKIIACLCNTAAFRMK

SEQ ID NO: 13 - protein, 47 aa, Bovine
LILLEVYIQFCSILGEAALKRQSKSIHFGQNFKIIACLCNTAAFRMK

SEQ ID NO: 14 - protein, 314 aa, Belgian Blue
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLVHFHTPPYGQWMFYRERKLILLEVYIQFCSILGEAALKRQSKSIHFGQNFKIIACLCNTAAFRMK

SEQ ID NO: 15 - DNA, 576 bp, Ovine
atggcgtgcggggcgacactgaagcggcccatggagttcgaggcggcgctgctgagccctggctctccgaagcggcggcgctgcgcccctctgtccggccccactccgggcctcaggcccccggacgccgaaccgccgccgctgcttcagacgcagaccccaccgccgactctgcagcagcccgccccgcccggcagcgagcggcgccttccaactccggagcaaatttttcagaacataaaacaagaatatagtcgttatcagaggtggagacatttagaagttgttcttaatcagagtgaagcttgtacttcggaaagtcagcctcactcctcagcactcacagcacctagttctccaggttcctcctggatgaaaaaggaccagcccacctttaccctccgacaagttggaataatatgtgagcgtctcttaaaagactatgaagataaaattcgggaggaatatgagcaaatcctcaatactaaactagcagaacaatatgaatcttttgtgaaattcacacatgatcagattatgcgacgatatgggacaaggccaacaagctatgtatcc

SEQ ID NO: 16 - protein, 192 aa, Ovine
MACGATLKRPMEFEAALLSPGSPKRRRCAPLSGPTPGLRPPDAEPPPLLQTQTPPPTLQQPAPPGSERRLPTPEQIFQNIKQEYSRYQRWRHLEVVLNQSEACTSESQPHSSALTAPSSPGSSWMKKDQPTFTLRQVGIICERLLKDYEDKIREEYEQILNTKLAEQYESFVKFTHDQIMRRYGTRPTSYVS

SEQ ID NO: 17 - DNA, 576 bp, Bovine
atggcgtgcggggcgacactgaagcggcccatggagttcgaggcggcgctgctgagccctggctctccgaagcgacggcgctgcgcccctctgtccggccccactccgggcctcaggcccccggacgccgaaccgccaccgctgcttcagacgcagatcccaccgccgactctgcagcagcccgccccgcccggcagcgaccggcgccttccaactccggagcaaatttttcagaacataaaacaagaatatagtcgttatcagaggtggagacatttagaagttgttcttaatcagagtgaagcttgtacttcggaaagtcagcctcactcctcaacactcacagcacctagttctccaggttcctcctggatgaaaaaggaccagcccacctttacgctccgacaagttggaataatatgtgagcgtctcttaaaagactatgaagataaaattcgggaggaatatgagcaaatcctcaatactaaactagcagaacaatatgaatcttttgtgaaattcacacatgatcagattatgcgacgatatgggacaaggccaacaagctatgtatcc

SEQ ID NO: 18 - protein, 192 aa, Bovine
MACGATLKRPMEFEAALLSPGSPKRRRCAPLSGPTPGLRPPDAEPPPLLQTQIPPPTLQQPAPPGSDRRLPTPEQIFQNIKQEYSRYQRWRHLEVVLNQSEACTSESQPHSSTLTAPSSPGSSWMKKDQPTFTLRQVGIICERLLKDYEDKIREEYEQILNTKLAEQYESFVKFTHDQIMRRYGTRPTSYVS

(19) United States
(12) Patent Application Publication
(10) Pub. No.: US 2008/0187543 A1
(43) Pub. Date: Aug. 7, 2008

(54) Use of Myostatin (Gdf-8) Antagonists for Improving Wound Healing and Preventing Fibrotic Disease

(75) Inventors: Ravi Kambadur, Hamilton (NZ); Mridula Sharma, Hamilton (NZ); Alex Hennebry, Hamilton (NZ); Monica Senna Salerno de Moura, Hamilton (NZ)

Correspondence Address: NIXON & VANDERHYE, PC, 901 NORTH GLEBE ROAD, 11TH FLOOR, ARLINGTON, VA, 22203, US

(73) Assignee: ORICO LIMITED, Dunedin (NZ)

(21) Appl. No.: 11/883,871

(22) PCT Filed: Feb. 7, 2006
(86) PCT No.: PCT/NZ06/00009

§ 371 Date: Nov. 6, 2007

(30) Foreign Application Priority Data
Feb. 7, 2005 (NZ) 538097

(51) Int. Cl. A61K 39/395; A61K 39/00; A61K 38/00; A61K 38/08; A61K 48/00; A61K 38/18; A61P 17/02

(52) U.S. Cl. 424/158.1; 424/198.1; 514/2; 514/17; 514/44

(57) ABSTRACT

The present invention relates to a method of improving wound healing in a human or animal patient by inhibiting the activity of myostatin (GDF-8) using one or more myostatin antagonists. The present invention also relates to a method of treating fibrotic diseases or disorders comprising administering a myostatin antagonist.

### FIELD OF THE INVENTION

The invention relates to methods and compositions for improving wound healing and in particular for preventing scar formation and thus loss of function that can occur in injured tissues during the natural wound healing process.

### BACKGROUND

A wound is a disruption of tissue integrity that is typically associated with a loss of biological substance. Simple wounds include cuts and scrapes to the skin whilst deeper injuries to the muscle tissue, skeletal system or the inner organs are defined as complicated wounds¹.

Every wound undergoes a similar reparative process no matter what the wound type or the degree of tissue damage^{1, 2, 3}. Three distinct phases of wound healing are recognised. Firstly the inflammatory or exudative phase for the detachment of deteriorated tissue and for wound cleansing; secondly a proliferative phase for the development of granulation tissue; and thirdly a differentiation or regeneration phase for maturation and scar formation¹.

The inflammatory phase is characterised by hemostasis and inflammation. After injury to tissue occurs, the cell membranes, damaged from the wound formation, release thromboxane A₂ and prostoglandin 2-alpha, potent vasoconstrictors. This initial response helps to limit haemorrhage. Capillary vasodilation then occurs and inflammatory cells (platelets, neutrophils, leukocytes, macrophages, and T lymphocytes), migrate to the wound site. In particular, neutrophil granulocytes play a central role in wound cleansing via phagocytosis. The next cells present in the wound are the leukocytes and macrophages. The macrophages in particular, are essential for wound healing. Numerous enzymes and cytokines are secreted by the macrophage, including collagenases, which debride the wound; interleukins and tumor necrosis factor (TNF), which stimulate fibroblasts (to produce collagen) and promote angiogenesis; and transforming growth factor (TGF), which stimulates keratinocytes². This step marks the transition into the process of tissue reconstruction, i.e. the proliferation phase.

The proliferation phase is characterised by epithelialisation, angiogenesis, granulation tissue formation, and collagen deposition. Angiogenesis stimulated by TNF alpha is essential to deliver nutrients into and around the wound site and is critical for efficient wound healing. Granulation tissue formation is a complex event involving leukocytes, histiocytes, plasma cells, mast cells, and in particular fibroblasts, that promote tissue growth through the production of collagen. The exact steps and mechanism of control of the proliferation phase are unknown. Some cytokines involved include platelet derived growth factor (PDGF), insulin like growth factor (IGF) and epidermal growth factor (EGF). All are necessary for collagen formation².

The final phase of wound healing is the differentiation phase. The wound undergoes contraction and the granulation tissue becomes increasingly depleted of fluids and blood vessels, begins to strengthen, and undergoes remodelling to form scar tissue. Where the wound involves damage to the skin, the final stage in wound healing is epithelialisation, whereby epidermal cells migrate to resurface the denuded area. Where a wound includes damage to skeletal muscle, new muscle cells are laid down (in addition to granulation tissue in the proliferative phase) via satellite cells which differentiate to form myoblasts⁴. In the final stage of wound healing the myoblasts differentiate to form myotubes which mature and are incorporated into muscle fibres. Whilst this process results in the gain of some muscle function at the wound site, muscle wounds invariably result in loss of muscle tissue, scarring and loss of original muscle function.

Current treatments for tissue wounds include methods of improving circulation and thus oxygen and nutrient delivery to a wound site to improve healing times. This may be achieved mechanically, such as by using ultrasound treatment, magnetic and electrical simulation, whirlpool therapy and oxygen therapy. However, whilst these therapies are effective in stimulating and even accelerating the wound healing process, they still result in functional and/or cosmetic impairment at the wound site⁵.

New therapies are currently being investigated using cytokines and growth factors such as TGF-beta, EGF and IGF-1. TNF agonists and antagonists may also be useful in modifying angiogenesis, thus providing significant potential to improve the healing process directly. However, to date growth factors have had a limited role in clinical practice. The only currently available commercial product is PDGF which has been shown to reduce healing time, but which has not been successful in improving the cosmetic or functional aspect of wound healing².

Thus, there is a need to provide new wound healing therapies which are able to control the wound healing process so that new tissue would replace damaged tissue with no functional or cosmetic impairment.

It is an object of the present invention to go some way towards fulfilling this need and/or to provide the public with a useful choice.

### SUMMARY OF THE INVENTION

Surprisingly, the growth factor myostatin, a member of the TGF-beta family of growth factors, has been shown for the first time to be implicated in the wound healing process. Inhibition of myostatin activity has been found to significantly improve the wound healing process.

Accordingly, the present invention provides a method of improving tissue wound healing comprising the step of administering an effective amount of at least one myostatin antagonist to a patient in need thereof. The invention may be useful in both animal and human wound healing.

Wound healing is improved in a human or animal patient via one or more of the following mechanisms:
(a) a decrease in the time of wound recovery;
(b) an acceleration and increase in the inflammatory response; and
(c) a decrease or inhibition of scar tissue formation,
   thereby resulting in improved functionality and cosmetic appearance of the treated tissue.

The myostatin antagonist may be selected from any one or more known myostatin inhibitors. For example, U.S. Pat. No. 6,096,506 and U.S. Pat. No. 6,468,535 disclose anti-myostatin antibodies. U.S. Pat. No. 6,369,201 and WO 01/05820 teach myostatin peptide immunogens, myostatin multimers and myostatin immunoconjugates capable of eliciting an immune response and blocking myostatin activity. Protein inhibitors of myostatin are disclosed in WO 02/085306, which include the truncated Activin type II receptor, the myostatin pro-domain, and follistatin. Other myostatin inhibitors derived from the myostatin peptide are known, and include for example myostatin inhibitors that are released into culture from cells overexpressing myostatin (WO 00/43781); dominant negatives of myostatin (WO 01/53350), which include the Piedmontese allele (cysteine at position 313 is replaced with a tyrosine) and mature myostatin peptides having a C-terminal truncation at a position either at or between amino acid positions 335 to 375. US2004/0181033 also teaches small peptides comprising the amino acid sequence WMCPP, and which are capable of binding to and inhibiting myostatin.

Preferably, the one or more myostatin antagonists comprise one or more dominant negatives selected from the group consisting of myostatin peptides that are C-terminally truncated at a position at or between amino acids 335, 350 and the Piedmontese allele.

The one or more myostatin antagonists may also include a myostatin splice variant comprising a polypeptide of any one of SEQ ID Nos: 8-14 or a functional fragment or variant thereof, or a sequence having 95%, 90% 85%, 80%, 75% or 70% sequence identity thereto.

The one or more myostatin antagonists may also include a regulator involved in the myostatin pathway comprising a polypeptide of SEQ ID No. 16 or SEQ ID No.18, or a functional fragment or variant thereof, or a sequence having at least 95%, 90%, 85%, 80%, 75% or 70% sequence identity thereto.

The myostatin antagonist may also include an anti-sense polynucleotide, an interfering RNA molecule, for example RNAi or siRNA, or an anti-myostatin ribozyme, which would inhibit myostatin activity by inhibiting myostatin gene expression.

When the one or more myostatin antagonists include an antibody, the antibody may be a mammalian or non-mammalian derived antibody, for example an IgNAR antibody derived from sharks, or the antibody may be a humanised antibody, or comprise a functional fragment derived from an antibody.

The present invention also provides for the use of one or more myostatin antagonists in the manufacture of a medicament for improving wound healing in a patient in need thereof.

The one or more myostatin antagonists may be selected from the group of myostatin antagonists disclosed above.

The medicament may be formulated for local or systemic administration, for example, the medicament may be formulated for topical administration to an external wound site, or may be formulated for injection to an internal wound site.

The present invention further provides a composition comprising one or more myostatin antagonists together with a pharmaceutically acceptable carrier, for use in a method of improving wound healing in a patient in need thereof.

The present invention further provides one or more myostatin antagonists for use in a method of improving wound healing in a patient in need thereof.

The invention will now be described in more detail with reference to the figures of the accompanying drawings in which:

### BRIEF DESCRIPTION OF THE FIGURES

FIG. 1A shows hematoxylin and eosin staining of control uninjured muscle sections from wild type and myostatin null mice;

FIG. 1B shows a low power view one day (D1) after wounding using notexin;

FIG. 1C shows a higher power view of the same sections as (B) stained to show eosinophilic (e) cytoplasm and fine intracellular vacuolation (v) of the myofibers with an increase in the intracellular spaces and marked myofiber disruption (arrows);

FIG. 1D shows day 2 (D2) muscle sections, with increased numbers of nuclei in muscle of myostatin null mice (arrows). Arrow heads denote the myonuclei along the margins of the necrotic myofibers;

FIG. 1E shows day 3 (D3) muscle sections with infiltrating mononucleated cells in both wild type and myostatin null muscle, but with higher numbers in the myostatin null sections. The scale bar equals 10 μm;

FIG. 1F shows day 5 sections (D5), having an increased number of nuclei within the wounded area of myostatin null muscle sections;

FIG. 2A shows the percentage of MyoD positive myogenic precursor cells in wild type (Mstn^{+/+}) and myostatin null (Mstn^{-/-}) regenerating muscle;

FIG. 2B shows the percentage of Mac-1 positive cells in wild type (Mstn^{+/+}) and myostatin null (Mstn^{-/-}) regenerating muscle;

FIG. 2C shows the expression profiles of MyoD and myogenin genes in control uninjured muscle (C) and regenerating wild type (wt) and myostatin null (Mstn null) muscle up to 28 days after wounding with notexin. GAPDH was used as a control to show equal amount of RNA used;

FIG. 3 Immunofluorescence on tissue sections obtained from myostatin knock-out (KO) and wild-type (WT) mice at day 14, 21 and 28 after injury WT tissue show stronger intensity of staining i.e. a higher concentration of vimentin positive cells when compared with KO tissue.

FIG. 4 shows the average number of Mac1 positive cells in recovering muscle 2, 3, 7 and 10 days after wounding with notexin in saline treated and myostatin antagonist 350 treated mice (dominant negative myostatin peptide C-terminally truncated at amino acid 350);

FIG. 5 shows the chemo-inhibitory effect of myostatin on macrophage migration and recovery using a myostatin antagonist (dominant negative myostatin peptide C-terminally truncated at amino acid 350);

FIG. 6A shows the chemo-attractant effect of myostatin on ovine primary fibroblast;

FIG. 6B shows the chemo-inhibitory effect of myostatin on ovine primary myoblasts and recovery using a myostatin antagonist (dominant negative myostatin peptide C-terminally truncated at amino acid 350);

FIG. 7 shows photomicrographs low power (i) and high power (ii) of hematoxylin and eosin staining (H&E) and Van Geisen (iii) staining of day 28 (D28) wild type and myostatin null muscle sections. Thick connective tissue (arrows) is seen in wild type muscle sections (ii); collagen (arrows) is seen in the wild type muscle sections (iii), scale bar equals 10 μm; a scanning electron micrograph of wild type and myostatin null muscle is shown in (iv) after 24 days of regeneration; scale bar equals 120 μm;

FIG. 8 shows the effect on muscle weight of a myostatin antagonist (dominant negative myostatin peptide C-terminally truncated at amino acid 350) in mice recovering from muscle wounding using notexin;

FIGS. 9A-D show hematoxylin and eosin staining of muscle sections from mice recovering from muscle wounding using notexin at day 7 (A-saline treated; B-myostatin antagonist 350 treated) and at day 10 (C-saline treated; D-myostatin antagonist 350 treated). Asterisks show necrotic areas;

FIG. 10 shows the percentage of unregenerated [symbol] and regenerated [symbol] areas of the muscle sections of FIG. 9;

FIG. 11 shows the percentage of collagen formation in regenerating muscle 10 and 28 days after wounding with notexin in saline treated and myostatin antagonist 350 treated mice;

FIG. 12 shows the average fibre area of regenerated muscle fibres 28 days after wounding with notexin in saline treated and myostatin antagonist 350 treated mice;

FIG. 13 Gene Pax7 (A) and MyoD (B) protein levels (detected through western blotting) 1, 3, 7, 10 and 28 days after the wounding with notexin in saline (sal) and myostatin antagonist 350 treated TA muscles;

FIG. 14 shows an increased inflammatory response in wounded muscle 2 and 4 days after wounding and an increased muscle mass in recovered muscle (at 21 days); and

FIG. 15 shows a schematic model for the role of myostatin in skeletal muscle healing.

### DEFINITIONS

“Wound” as used throughout the specification and claims means damage to one or more tissue, and is not to be limited to open wounds, for example, cuts, scrapes, surgical incisions and the like, but also includes internal wounds, for example, bruises, haematomas and the like as well as burns.

“Inhibitor” or “antagonist” as used throughout the specification and claims means any compound that acts to decrease, either in whole or in part, the activity of a protein. This includes a compound that either binds to and directly inhibits that activity of the protein, or may act to decrease the production of the protein or increase its production, thereby affecting the amount of the protein present and thereby decreasing its activity.

“Gene expression” as used through the specification and claims means the initiation of transcription, the transcription of a section of DNA into mRNA, and the translation of the mRNA into a polypeptide.

“Comprising” as used throughout the specification and claims means ‘consisting at least in part of’, that is to say when interpreting independent claims including that term, the features prefaced by that term in each claim all need to be present but other features can also be present.

### DETAILED DESCRIPTION OF THE INVENTION

The present invention shows for the first time that myostatin is involved in the processes of wound healing. In particular, myostatin appears to be a negative regulator of all of the three characteristic phases of wound healing, i.e. the inflammatory phase, the proliferation phase, and the differentiation phase.

For example, when myostatin is absent, such as in myostatin null mice, or is inhibited, for example using a myostatin antagonist, there is an increase in the number of macrophages and an earlier migration of macrophages to the wound site (inflammatory phase), less collagen is deposited (proliferation phase) and there is a significant reduction in scar tissue formation (differentiation phase).

Thus, myostatin appears to be a powerful regulator of the wound healing process and can be manipulated to prevent scar formation and resulting loss of function that would normally occur in injured tissue during the natural wound healing process. Lack of scarring is also important for cosmetic purposes, especially when the wound affects external portions of the body which are easily seen, such as the face, neck, hands etc.

The present invention is thus directed to a method of improving tissue wound healing comprising the step of administering an effective amount of at least one myostatin antagonist to a patient in need thereof. The patient is preferably a human patient, but the method of the present invention may also be used to improve wound healing in non-human animals.

Wound healing is improved in a human or animal patient via one or more of the following mechanisms:
(d) a decrease in the time of wound recovery;
(e) an acceleration and increase in the inflammatory response; and
(f) a decrease or inhibition of scar tissue formation,
   thereby resulting in improved functionality and cosmetic appearance of the treated tissue.

The myostatin antagonist may be selected from one or more molecules that are capable of inhibiting, in whole or in part, the activity of myostatin.

In particular, myostatin antagonist may be selected from any one or more known myostatin inhibitors. For example, U.S. Pat. No. 6,096,506 and U.S. Pat. No. 6,468,535 disclose anti-myostatin antibodies. U.S. Pat. No. 6,369,201 and WO 01/05820 teach myostatin peptide immunogens, myostatin multimers and myostatin immunoconjugates capable of eliciting an immune response and blocking myostatin activity. Protein inhibitors of myostatin are disclosed in WO 02/085306, which include the truncated Activin type II receptor, the myostatin pro-domain, and follistatin. Other myostatin inhibitors derived from the myostatin peptide are known, and include for example myostatin inhibitors that are released into culture from cells overexpressing myostatin (WO 00/43781); dominant negatives of myostatin (WO 01/53350), which include the Piedmontese allele (cysteine at position 313 is replaced with a tyrosine) and mature myostatin peptides having a C-terminal truncation at a position either at or between amino acid positions 335 to 375. US2004/0181033 also teaches small peptides comprising the amino acid sequence WMCPP, and which are capable of binding to and inhibiting myostatin.

Preferably, the myostatin antagonist is a dominant negative peptide. These are peptides derived from a parent protein that act to inhibit the biological activity of the parent protein. As mentioned above, dominant negative peptides of myostatin are known and include a mature myostatin peptide that is C-terminally truncated at a position at or between amino acids 335, 350 and the Piedmontese allele (wherein the cysteine at position 313 is replaced with a tyrosine).

Myostatin is known to be involved in myogenesis and is a negative regulator of muscle growth^{6,7}. Myostatin is initially produced as a 375 amino acid precursor molecule having a secretary signal sequence at the N-terminus, which is cleaved off to leave an inactive pro-from. Myostatin is activated by furin endoprotease cleavage at Arg 266 releasing the N-terminal pro-domain (or latency-associated peptide (LAP) domain) and the mature myostatin domain. However, after cleavage, the pro-domain can remain bound to the mature domain in an inactive complex⁸. Therefore, the pro-domain, or fragments thereof, can also be used in the present invention as a myostatin antagonist to improve wound healing.

A splice variant of myostatin has been identified which also acts as a myostatin antagonist (PCT/NZ2005/000250). The myostatin splice variant (MSV) results from an extra splice event which removes a large portion of the third exon. The resulting MSV polypeptide, ovine (oMSV; SEQ ID No: 8) and bovine MSV (bMSV; SEQ ID No: 11) shares the first 257 amino acids with native myostatin propeptide, but has a unique 64 amino acid C-terminal end (ovine oMSV65, SEQ ID No: 9 and bovine bMSV65, SEQ ID No: 12). The mRNA differs by 195 nucleotides, however, the valine residue at position 257 in MSV is the same as the canonical myostatin sequence. The MSV of the Belgian Blue cattle (bMSVbb; SEQ ID No: 7) encodes for a 7aa shorter 314aa protein (SEQ ID No: 14) but the rest of the protein sequence shows complete homology in the two breeds examined. The unique 65aa C-terminal peptide (SEQ ID No: 12) is conserved in bMSVbb.

It has also been discovered that a (KERK) cleavage site, for propeptide convertase (PC1-7) which includes furin endopeptidase, exists at position 271 to 274. Cleavage at position 274, releases a 47 amino acid C-terminal mature MSV fragment (ovine oMSV47, SEQ ID No: 10 and bovine bMSV47, SEQ ID No: 13).

The 65 amino acid MSV fragment (SEQ ID NO:12) has been shown to act as a myostatin antagonist in vitro (PCT/NZ2005/000250) and it is expected that MSV in vivo will act to regulate myostatin activity. Therefore, the MSV polypeptides disclosed herein could be used to inhibit myostatin the therefore promote wound healing according to the present invention.

Another myostatin antagonist is a modulator of myostatin gene expression. The myostatin gene expression may be altered by introducing polynucleotides that interfere with transcription and/or translation. For example, anti-sense polynucleotides could be introduced, which may include; an anti-sense expression vector, anti-sense oligodeoxyribonucleotides, anti-sense phosphorothioate oligodeoxyribonucleotides, anti-sense oligoribonucleotides, anti-sense phosphorothioate oligonucleotides, or any other means that is known in the art, which includes the use of chemical modifications to enhance the efficiency of anti-sense polynucleotides. Antisense molecules of myostatin may be produced by methods known in the art⁹ and by knowledge of the myostatin gene sequence^{6,7}.

It will be appreciated that any anti-sense polypeptide need not be 100% complementary to the polynucleotides in question, but only needs to have sufficient identity to allow the anti-sense polynucleotide to bind to the gene, or mRNA to disrupt gene expression, without substantially disrupting the expression of other genes. It will also be understood that polynucleotides that are complementary to the gene, including 5′ untranslated regions may also be used to disrupt translation of the myostatin protein. Likewise, these complementary polynucleotides need not have 100% complementary, but be sufficient to bind the mRNA and disrupt translation, without substantially disrupting the translation of other genes.

The modulation of gene expression may also comprise the use of an interfering RNA molecule including RNA interference (RNAi) or small interfering RNA (siRNA), as would be appreciated by a skilled worker by following known techniques¹⁰.

Modulation of gene expression may also be achieved by the use of catalytic RNA molecules or ribozymes. It is known in the art that such ribozymes can be designed to pair with a specifically targeted RNA molecule. The ribozymes bind to and cleave the targeted RNA¹¹.

Any other techniques known in the art of regulating gene expression and RNA processing can also be used to regulate myostatin gene expression.

A further antagonist of myostatin is a peptide derived from myostatin receptors. Such, receptor derived fragments generally include the myostatin binding domain, which then binds to and inhibits wildtype myostatin. The myostatin receptor is activin type IIB and its peptide sequence is known⁸. Thus, a skilled worker could produce such receptor antagonists without undue experimentation.

Another myostatin antagonist includes an anti-myostatin antibody. Antibodies against myostatin are known in the art, as described above, as are methods for producing such antibodies. The antibody may be a mammalian or a non-mammalian antibody, for example the IgNAR class of antibodies from sharks; or a fragment or derivative derived from any such protein that is able to bind to myostatin.

It will be appreciated that other molecules involved in the myostatin signalling pathway will be suitable for use in the present invention, particularly molecules that have an antagonistic action to myostatin. One such peptide, known as “mighty”, disclosed in PCT/NZ2004/000308, acts to promote muscle growth. “Mighty” expression is repressed by myostatin and therefore is involved in the same signalling pathway. Therefore it will be appreciated that instead of directly inhibiting myostatin, a peptide which opposes the signalling action of myostatin, for example “mighty”, could be used to promote wound healing.

It is anticipated that a polynucleotide that encodes the “mighty” gene (ovine; SEQ ID No:15 and bovine; SEQ ID No: 17) could be used for localised gene therapy at the wound site, having either permanent or transient expression of “mighty”, or alternatively the “mighty” protein (ovine; SEQ ID No.16 and bovine; SEQ ID No.18) could be used directly. It will be appreciated that due to the redundancy in the genetic code sequences that have essentially the same activity can be produced that are not identical to those disclosed in SEQ ID Nos: 15-18. Furthermore peptides having changes in none critical domains that have the same essential function can also be created. Changes can include insertions, deletions, or changes of one amino acid residue to another. Such variations are encompassed within the scope of the present invention.

The present invention is based on the finding that myostatin is able to promote wound healing or ameliorate wound damage. Wound healing is improved in a human or animal patient via one or more of the following mechanisms:
(g) a decrease in the time of wound recovery;
(h) an acceleration and increase in the inflammatory response; and
(i) a decrease or inhibition of scar tissue formation,
   thereby resulting in improved functionality and cosmetic appearance of the treated tissue. Therefore any myostatin antagonist, known or developed, is suitable for use in the method of the invention. This includes any molecule capable of binding to myostatin, for example, a IMM7 immunity protein from E. coli, or any other class of binding protein known in the art. Other peptides that can bind and inhibit myostatin are known, for example, peptides containing the amino acids WMCPP (US2004/0181033). It will be appreciated that any compound that is capable of inhibiting myostatin will be useful in the method and medicaments of the present invention.

Myostatin is a secreted growth factor that is mainly synthesised in skeletal muscle. However, myostatin is also present in other tissues including heart, mammary gland, adipose tissue and brain, and the myostatin receptor is ubiquitous. It is therefore expected that myostatin antagonists will be effective in promoting wound healing in tissues where myostatin is present or the myostatin receptor is present, or in organs, such as skin, comprising such tissues.

The myostatin antagonists, useful in the method of the present invention, may be tested for biological activity in an animal model or in vitro model of wound healing as discussed below and suitably active compounds formulated into pharmaceutical compositions. The pharmaceutical compositions of the present invention may comprise, in addition to one or more myostatin antagonists described herein, a pharmaceutically acceptable excipient, carrier, buffer, stabiliser or other material well known in the art. Such materials should be non-toxic and should not interfere with the efficacy of the active ingredient. The precise nature of the carrier or other material will be dependent upon the desired nature of the pharmaceutical composition, and the route of administration e.g. oral, intravenous, cutaneous, subcutaneous, intradermal, topical, nasal, pulmonary, intramuscular or intraperitoneal.

Pharmaceutical compositions for oral administration may be in tablet, lozenge, capsule, powder, granule or liquid form. A tablet or other solid oral dosage form will usually include a solid carrier such as gelatine, starch, mannitol, crystalline cellulose, or other inert materials generally used in pharmaceutical manufacture. Similarly, liquid pharmaceutical compositions such as a syrup or emulsion, will generally include a liquid carrier such as water, petroleum, animal or vegetable oils, mineral oil or synthetic oil.

For intravenous, cutaneous, subcutaneous, intradermal or intraperitoneal injection, the active ingredient will be in the form of a parenterally acceptable aqueous solution which is pyrogen-free and has suitable pH, isotonicity and stability.

For topical administration, the active ingredient will be dissolved or suspended in a suitable emollient and may be formulated in the form of a cream, roll-on, lotion, stick, spray, ointment, paste, or gel, and can be applied directly to the wound site or via a intermediary such as a pad, patch or the like.

For nasal or pulmonary administration, the active ingredients will be in the form of a fine powder or a solution or suspension suitable for inhalation. Alternatively, the active ingredients may be in a form suitable for direct application to the nasal mucosa such as an ointment or cream, nasal spray, nasal drops or an aerosol.

A particularly preferred application of the myostatin antagonists described herein is in the treatment of muscle wounds.

The ability of one or more myostatin antagonists to treat muscle wounds can be demonstrated in a notexin model of muscle injury as previously described¹².

Another preferred application of the present invention is in the treatment of skin wounds.

The ability of one or more myostatin antagonists to treat superficial or deep skin wounds can be demonstrated according to known methods¹³.

Another preferred application in the present invention is in the treatment of burns.

The ability of one or more myostatin antagonists to treat burn wounds can be demonstrated in known animal models. For example as described in Yang et al¹⁴.

In a further embodiment, the invention contemplates the use of one or more additional immuno-responsive compounds co-administered with the pharmaceutical composition of the present invention to give an additive or synergistic effect to the treatment regime. Such an immuno-responsive compound will generally be an immune response inducing substance. Examples of such substance include glucocorticosteroids, such as prednisolone and methylprednisolone; nonsteroidal anti-inflammatory drugs (NSAIDs); PDGF, EGF, IGF, as well as first and second generation anti-TNFα agents. Such substances may be administered either separately, sequentially or simultaneously with at least one myostatin antagonist described herein depending upon the type of wound to be treated as will be appreciated by a skilled worker. Administration of the pharmaceutical composition of the invention is preferably in a “prophylactically effective amount” or a “therapeutically effective amount”, this being sufficient to show benefit to the individual. The actual amount administered, and rate and time-course of administration, will depend on the nature and severity of the type of wound that is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16^{th} edition, Oslo, A. (ed), 1980.

The present invention is also directed to the use of one or more myostatin inhibitors in the manufacture of a medicament for improving wound healing in a patient in need thereof. The one or more myostatin antagonists may be selected from the group of myostatin antagonists described above.

The medicament may be formulated for local or systemic administration, for example, the medicament may be formulated for topical administration to an external or open wound site, or may be formulated for injection into an internal or deep wound site.

The medicament may further comprise one or more additional immuno-responsive compounds to give an additive or synergistic effect on wound healing, selected from the group of immuno-responsive compounds described above. The medicament may be formulated for separate, sequential or simultaneous administration of one or more myostatin antagonists and the one or more immuno-reactive compounds.

Without being bound by theory, it is thought that myostatin antagonists are effective in improving wound healing by acting at all three recognised phases of wound healing, i.e. the inflammatory phase, the proliferation phase and the differentiation phase described above.

For example, inhibition of myostatin activity is shown for the first time to have a direct effect on macrophage recruitment. In particular, both the number of macrophages and the migration time to the wound site are increased when myostatin is either absent (in myostatin null mice), or is inhibited, using a myostatin antagonist. Thus, the first phase of wound healing, the inflammatory phase, is significantly improved and it is expected that this will result in faster and more efficient wound cleansing and angiogenesis.

In addition, inhibition of myostatin activity, has also been shown to result in less collagen being deposited in the proliferation phase. Myostatin is shown here for the first time to be a chemo-attractant for fibroblasts. Thus, inhibition of myostatin activity is thought to result in the recruitment of less fibroblasts to the wound site and thus less production of collagen by the reduced population of fibroblasts.

Myostatin is further shown for the first time to be involved in scar tissue formation in the differentiation phase of wound healing. Specifically, inhibition of myostatin activity has been shown to result in a significant reduction in scar tissue formation in a recovered wounded tissue. In addition, there was also a significant reduction in loss of functional tissue, i.e. myostatin inhibition also resulted in improved tissue regeneration, so that the recovered tissue was replaced without scarring and thus had little functional or cosmetic impairment. This may be particularly beneficial in cosmetic surgery or in treating wounds to portions of the body that are clearly visible, such as face, neck, hands etc.

Whilst the present invention is exemplified in models of muscle wounds only, it is expected that, it would work equally well with other types of wounds such as skin cuts and abrasions, deep wounds extending through the skin and muscle (including surgical incisions) as well as internal wounds (for example wounds to muscle and tendon caused by sports injury or trauma), bruises, hematomas, and burns.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### EXAMPLES

### Example 1

### Myostatin Antagonists Increase Inflammatory Response and Chemotaxis of Cells Involved in Muscle Wound Healing

Wound healing is a highly ordered process; muscle tissue wounding results in immediate inflammatory response followed by chemotactic movement of myogenic precursor satellite cells. Here we have shown that myostatin actually inhibits the inflammatory response and the chemotactic movement of myogenic cells towards the wound site. Thus the beneficial effects of lack of myostatin or antagonists of myostatin on the speed and quality of wound healing are demonstrated.

### Materials and Methods

### Expression and Purification of 350

A cDNA corresponding to the 267-350 amino acids, of bovine myostatin (hereafter referred to as “350” or “350 protein”) was PCR amplified and cloned into pET16-B vector. Expression and purification of 350 protein was done according to the manufacturer's (Qiagen) protocol under native conditions.

### Notexin Wounding Model

Six to eight week old male C57BL/10 and Mstn^{-/-} mice (n=27 per group) were anaesthetized, using a mixture of 25% Hypnorm (Fentanyl citrate 0.315 mg/ml and Fluanisone 10 mg/ml) and 10% Hypnovel (Midazolam at 5 mg/ml) at 0.1 ml/10 g body weight. The tibialis anterior muscle of the right leg was injected intramuscular with 10 μl of 10 μg/ml Notexin, using a 100 μl syringe (SGE, Australia). Tibialis anterior muscles were removed from euthanized mice at day 0 (control), and days 1, 2, 3, 5, 7, 10, 14 or 28 (n=3 per day). The tibialis anterior muscles were mounted in Tissue Tec and frozen in isopentane chilled in liquid nitrogen. For trials of 350 on wounding, 1 year old wild type mice were injected with notexin as mentioned above into the left tibialis anterior (TA) muscle. Wounded mice were either injected subcutaneously with the myostatin antagonist, 350, at 6 μg per gram of body weight, or the equivalent amount of saline (control mice) on days 1, 3, 5, and 7. To assess the effect of 350 on muscle healing, mice were euthanized on days 1, 3, 7, 10 and 28 after injection of notexin and TA muscles were dissected out and processed for protein isolation or tissue sectioning. Frozen muscle samples were stored at −80° C. Seven μm transverse sections (n=3) were cut at 3 levels, 100 μm apart. The sections were then stained with hematoxylin and eosin or Van Geisen. Sections were then examined and photographed using an Olympus BX50 microscope (Olympus Optical Co., Germany) fitted with a DAGE-MTI DC-330 colour camera (DAGE-MTI Inc.).

### Immunohistochemistry

Frozen muscle sections (7 μm thick) were post fixed in 2% paraformaldehyde and then permeabilised in 0.3% (v/v) Triton X-100 in PBS and then blocked with 10% (v/v) normal goat serum-Tris buffered saline (NGS-TBS) for 1 hour at RT. The sections were incubated with antibodies diluted in 5% NGS-TBS overnight at 4° C. The antibodies used were mouse anti-MyoD, 1:25 dilution (554130; PharMingen) a specific marker for activated myoblasts (Cooper et al., 1999; Koishi et al., 1995); goat anti-Mac-1, 1:400 dilution (Integrin M-19; Santa Cruz) an antibody specific for infiltrating peripheral macrophages¹⁵; mouse anti-vimentin antibody at 1:300 dilution a marker for fibroblasts. The sections were washed 3 times with PBS, then were incubated with either donkey anti-mouse Cy3 conjugate, 1:400 dilution (715-165-150; Jackson ImmunoResearch, West Grove, Pa., USA) or biotinylated donkey anti-sheep/goat IgG antibody 1:400 dilution (RPN 1025; Amersham). Secondary antibody incubation was followed by incubation with streptavidin conjugated to fluorescein, 1:400 dilution (S-869; Molecular Probes) diluted in 5% NGS-TBS for 30 min at RT. Sections were rinsed with PBS 3 times, counter stained with DAPI and mounted with Dako® fluorescent mounting medium. Tibialis anterior muscle sections were examined by epi-fluorescent microscopy. Representative micrographs were taken on an Olympus BX50 microscope (Olympus Optical Co., Germany) fitted with a DAGE-MTI DC-330 colour camera (DAGE-MTI Inc., 1N, USA). The average muscle area was measured using the Scion Imaging program (NIH) with 5 random muscle sections used previously for immunohistochemistry from Mstn^{-/-} and wild type mice.

### Chemotaxis Assay

Primary myoblasts were cultured from the hind limb muscle of 4 to 6 week old mice, according to the published protocols^{16,17}. Briefly, muscles were minced, and digested in 0.2% collagenase type 1A for 90 min. Cultures were enriched for myoblasts by pre-plating on uncoated plates for 3 hours. Myoblast cultures were maintained in growth media (GM) supplemented with 20% fetal calf serum (FCS), 10% HS and 1% CEE on 10% Matrigel coated plates, at 37° C./5% CO₂. The extent of culture purity was assessed by flow cytometry analysis of MyoD expression after 48 hours in culture. Cells were harvested using trypsin, suspended at a concentration of 10⁶ cells/200 μl and fixed overnight in 5 ml 70% ethanol at −20° C. Staining was performed for 30 min at room temperature using rabbit polyclonal anti-MyoD, 1:200 (Santa Cruz), followed by Alexa fluor 488 anti-rabbit conjugate, 1:500 (Molecular Probes). Analysis was carried out in duplicate with 10⁴ cell events collected in each assay. Debris was excluded by gating on forward and side scatter profiles. Cells were analyzed by FACScan (Becton Dickinson). Macrophages were isolated by a peritoneal lavage technique. Zymosan-activated mouse serum (ZAMS) was prepared according to the published protocol (Colditz and Movat, 1984). Chemotaxis experiments were performed in single blind-well Boyden-type chambers with 7 mm diameter wells (Neuro Probe, MD USA). Standard polycarbonate filters with 8 μm holes (Neuro probe; holes=6% of surface area) were washed thoroughly, and for the myoblast assay, filters were treated with 1% Matrigel in DMEM for 30 min. Filters were then dried and placed between the top and bottom chambers.

For the chemotaxis assay of myoblasts, DMEM containing 5% chicken embryo extract (CEE) plus dialysis buffer was used as positive control. Recombinant myostatin (2.5 and 5 μg/ml myostatin) and 350 protein (at 5-times myostatin concentration, i.e., 12.5 μg/ml and 25 μg/ml) were added to positive control medium, Plain DMEM was used as negative control. On a 24-well plate, the bottom wells were filled with test or control media. Seventy-five thousand cells were added to the top wells. The plate was incubated for 7 h at 37° C., 5% CO₂. The top surface of the membranes was washed with pre-wet swabs to remove cells that did not migrate. The membrane was then fixed, stained in Gill's hematoxylin and wet mounted on slides. Migrated cells were counted on four representative fields per membrane and the average number plotted.

For chemotaxis assay of macrophages, DMEM containing 33% Zymosan-activated mouse serum (ZAMS) plus dialysis buffer was used as positive control. Recombinant myostatin (5 μg/ml myostatin) and 350 protein (at 2 and 5-times myostatin concentration, i.e., 10 μg/ml and 25 μg/ml) were added to positive control medium or plain DMEM. On a 24-well plate, the bottom wells were filled with test or control media. Seventy-five thousand cells were added to the top wells containing polyethylene terephthalate (PET) 0.8 μm membranes. The plate was incubated for 4 h at 37° C., 5% CO₂. The top surface of the membranes was washed with pre-wet swabs to remove cells that did not migrate. The membrane was then fixed, stained in Gill's hematoxylin and wet mounted on slides. Migrated cells were counted on four representative fields per membrane and the average number plotted.

Primary fibroblasts were obtained from lamb skin explants. DMEM containing 10 pg/ml of recombinant TGF-β was used as positive control. Recombinant myostatin (5 μg/ml myostatin) was added to positive control media. On a 24-well plate, the bottom wells were filled with test or control media. Eighty eight thousand cells were added to the top wells containing polyethylene terephthalate (PET) 0.8 μm membranes. The plate was incubated for 4 h at 37° C., 5% CO₂. The top surface of the membranes was washed with pre-wet swabs to remove cells that did not migrate. The membrane was then fixed, stained in Gill's hematoxylin and wet mounted on slides. Migrated cells were counted on four representative fields per membrane and the average number plotted.

### RT PCR for Gene Expression

Total RNA was isolated using Trizol (Invitrogen) according to the manufacturer's protocol. Reverse transcription reaction was performed using Superscript preamplification kit (Invitrogen). PCR was performed with 1 μl of the reverse transcription reaction, at 94° C. for 30 s, 55° C. for 30 s, and 72° C. for 30 s. For each gene, number of cycles required for exponential amplification was determined using varying cycles. The amplicons were separated on an agarose gel and transferred to a nylon membrane. The PCR products were detected by Southern blot hybridization. Each data point was normalized by the abundance of glyceraldhyde-3-phosphate dehydrogenase (GAPDH) mRNA.

### Results

### Myostatin Influences the Chemotaxis of Myoblasts, Macrophages and Fibroblasts.

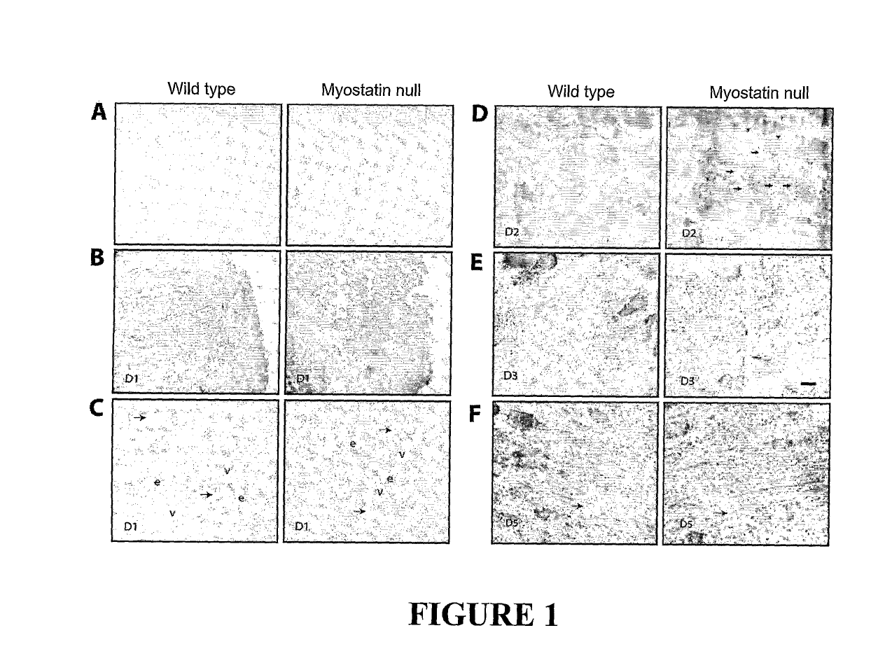

Inflammatory response to muscle wounding, as shown by the presence of eosinophils, and myoblast migration was seen within 24 hours after notexin wounding in both wild type and Mstn^{-/-} muscle (FIG. 1C). By day 2, the differences between wild type and Mstn^{-/-} responses in inflammatory response and satellite cell migration were pronounced with a marked increase in accretion of nuclei at the site of wounding in Mstn^{-/-} muscle sections (FIG. 1D, arrows). Increased numbers of nuclei observed are due to increased numbers of macrophages and myoblasts. The highest density of nuclei was seen along the margins of the necrotic myofibers (FIG. 1D, arrowheads), particularly in Mstn^{-/-} sections. By day 3 recovering wild type muscle sections also showed an increase in number of nuclei, although still far less than in comparable tissue collected from the Mstn^{-/-} mice (FIG. 1E). Accretion of mononuclear cells following notoxin wounding peaked at day 5 in both wild type and Mstn^{-/-} muscle sections (FIG. 1F). The major effect noted was an accelerated migration of macrophages and myoblasts to the wound site in Mstn^{-/-} muscle sections.

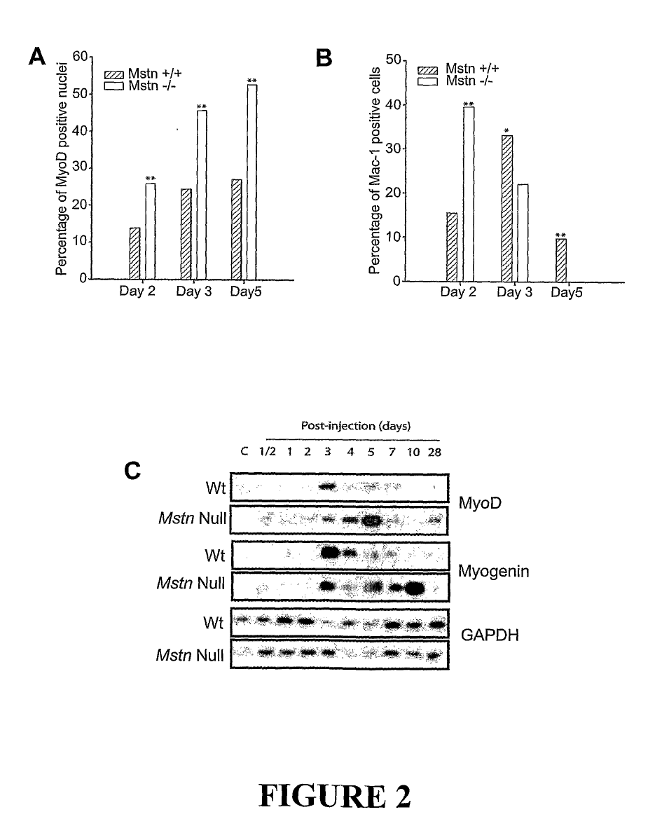

In response to muscle wounding inflammatory cells and satellite cells migrate to the site of wounding¹⁸. To determine if lack of myostatin enhances the migration of either activated satellite cells or inflammatory cells, the proportion of the inflammatory cells and myoblasts at the site of wounding was quantified. Immunohistochemistry was used to detect MyoD, a specific marker for myoblasts¹⁹, and Mac-1, for infiltrating peripheral macrophages²⁰. Control untreated muscle sections were found to be negative for MyoD immunostaining. Muscle sections were stained with DAPI to count total number of nuclei. Quantification results demonstrate that in the Mstn^{-/-} recovering muscle, twice the number of myogenic cells (MyoD positive) (FIG. 2A) and macrophages (Mac-1 positive) (FIG. 2B) are present at the site of wound healing at day 2 compared to the wild type sections. From day 2 through to day 5 of wound healing, Mstn^{-/-} muscle sections had more myoblasts than wild type muscle (FIG. 2A). Like the MyoD positive cells, the increased infiltration of macrophages to the site of wounding was seen much earlier (on day 2) in the Mstn^{-/-} muscle in response to wounding (FIG. 2B). In addition, the inflammatory cell numbers decreased more rapidly in the Mstn^{-/-} muscle indicating that the whole process of inflammatory cell response was accelerated in Mstn^{-/-} mice (FIG. 2B).

Grounds et al²¹ demonstrated that MyoD and myogenin gene expression can be used as markers for the very early detection of migrating myoblasts during muscle wound healing. Hence the expression of MyoD and myogenin was determined in the recovering tissue. Quantitative RT-PCR results confirm that the expression of the muscle regulatory factors myoD and myogenin, were expressed earlier in Mstn^{-/-} muscle as compared to wild type muscle. High levels of MyoD mRNA were detected within 12 hours after wounding in the Mstn^{-/-} muscle. In the wild type muscle however, MyoD expression was un-detectable until day 1 after wounding (FIG. 2C). Similarly, higher levels of mRNA for myogenin, was also detected very early within 12 hours after wounding in the regenerating Mstn^{-/-} muscle. However, in the wild type recovering muscle, myogenin mRNA was not detected until 1 day after the muscle wounding (FIG. 2C). Thus results from immunohistochemistry and gene expression analysis concur that there is increased and hastened migration of myogenic cells to the site of wounding in Mstn^{-/-} muscle.

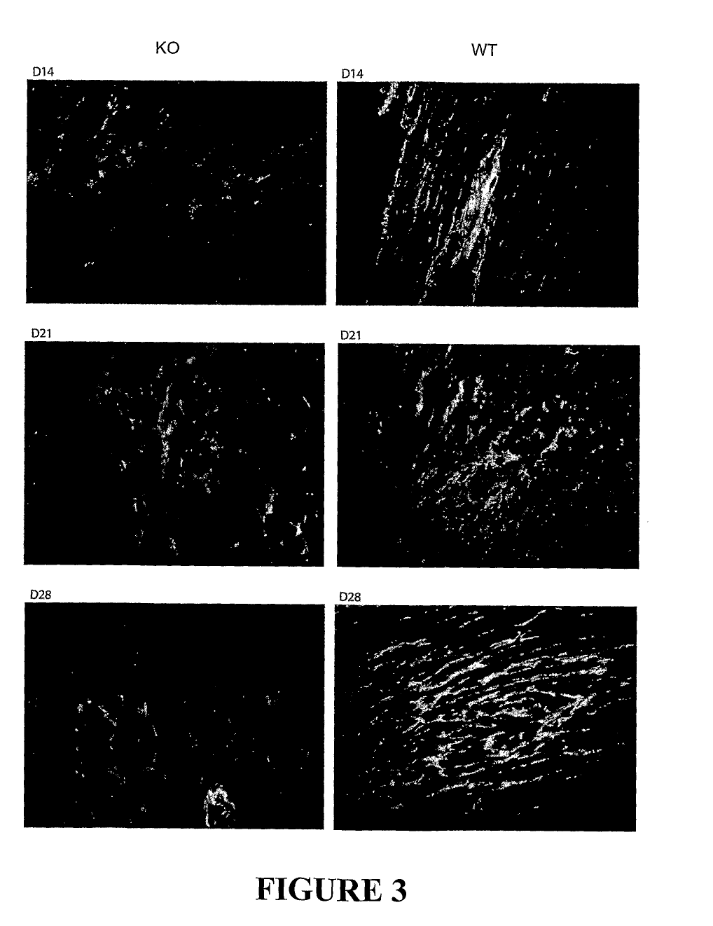

In addition to myoblasts, fibroblasts also migrate and populate the wound site. The effect of myostatin on the dynamics of fibroblast migration during muscle wound healing was investigated. As shown in FIG. 3 staining with vimentin antibody (a specific marker for fibroblasts) indicate that there is substantially less accretion of fibroblasts in the TA muscles in Mstn^{-/-} mice at the wound site as compared to wild type muscle. This result, in combination with data below on migration assays on fibroblasts, clearly demonstrates that myostatin acts as a chemoattractant for fibroblasts.

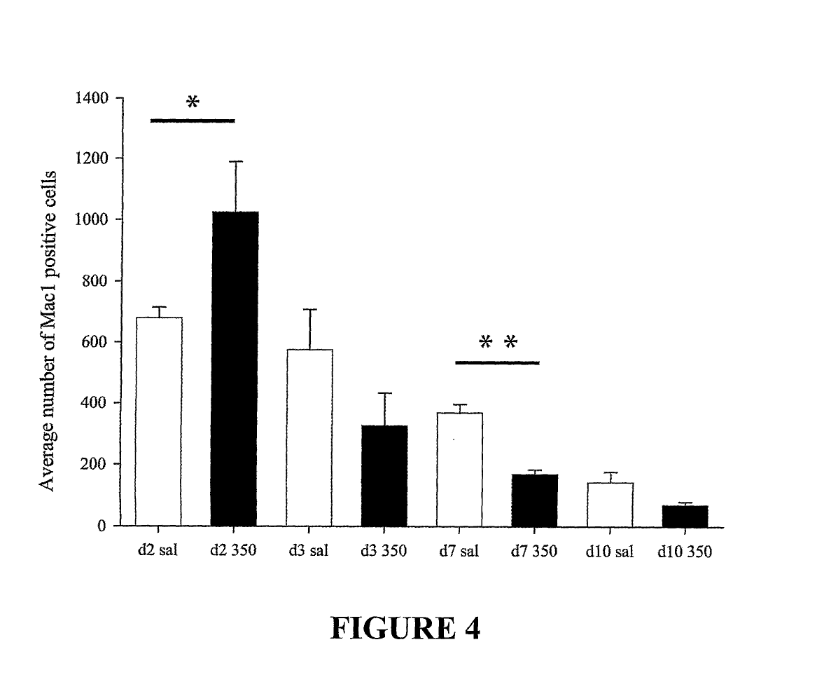

To demonstrate the beneficial effects of myostatin activity inhibition by 350 on enhanced inflammatory response, mice undergoing wound healing after notexin wounding were treated with 350 protein and the inflammatory response was determined. A greater percentage of Mac1 positive macrophages were found in day 2 injured muscles which had been treated with 350 (FIG. 4). By day 3, the percentage had dropped in the 350 treated muscles below that of the saline treated day 3 muscles and continued to be lower in day 7 and 10 muscles. This result indicates an early or more profound recruitment of macrophages in the 350 treated muscles by day 2, followed by a decreased recruitment by day 7 and 10. These results show accelerated wound healing processes with the 350 treatment.

### Inhibition of Chemotaxis of Myoblasts and Macrophages by Myostatin and its Rescue by 350

It has been demonstrated that there is a three-fold increase in myostatin levels in thermally wounded tissues (burns) at 24 hrs after wounding¹⁴. Similarly, in muscle tissues wounded by notexin a significant increase in myostatin levels was measured in muscle tissue at 24 hrs after wounding¹².

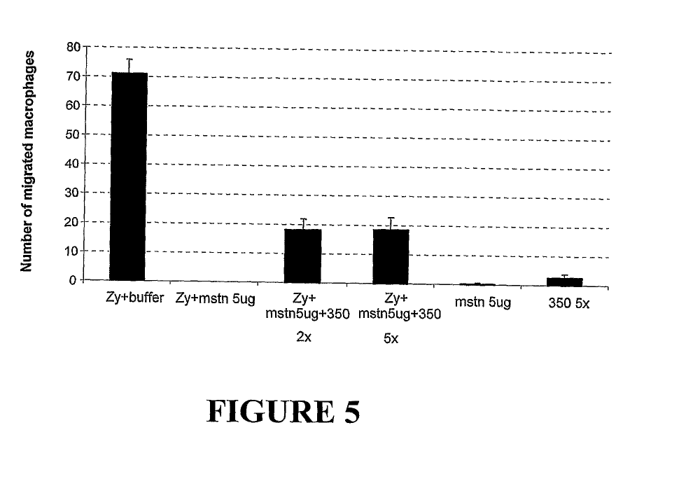

Results presented above indicate that Mstn^{-/-} muscle has an increased and accelerated infiltration of macrophages and migration of myoblasts to the area of wounding. Since both cell types are known to be influenced by chemotactic factors to direct their movement^{22, 23} the effect of myostatin on the migratory ability of satellite cell derived myoblasts and macrophages was investigated. To test whether myostatin interferes with chemotactic signals, blind-well chemotaxis chambers were used. Isolated myoblasts or macrophages were assessed for their migratory ability through a filter towards a chemo-attractant (CEE for myoblasts, and ZAMS activated serum for macrophages). The isolated myoblasts were found to be 90% myogenic (MyoD positive) as assessed by flow cytometry. As shown in FIG. 5, addition of 5 μg/ml myostatin to ZAMS medium completely abolishes macrophage migration. When 350 protein is added to the medium containing 5 μg/ml myostatin, a significant rescue of the chemo-inhibitory effect of myostatin on macrophages is observed (20-fold increase). This result confirms that administration of myostatin inhibitors such as 350 can accelerate wound healing by decreasing the inhibition of macrophage migration by myostatin.

In addition to the effects on macrophage migration, here we also demonstrate that myostatin antagonists such as 350 can also decrease the negative effects of myostatin on the chemotactic movement of myoblasts. Addition of recombinant myostatin at 2.5 and 5 μg/ml to positive control medium leads to 66 and 82% inhibition of myoblast migration respectively. When 350 protein is added to the medium containing recombinant myostatin, the chemo-inhibitory effect of myostatin on myoblasts is rescued to levels similar to observed in the positive control thus demonstrating that myostatin antagonists such as 350 can effectively accelerate wound healing by enhancing myoblast migration.

### Myostatin Acts as a Chemo-Attractant for Fibroblasts

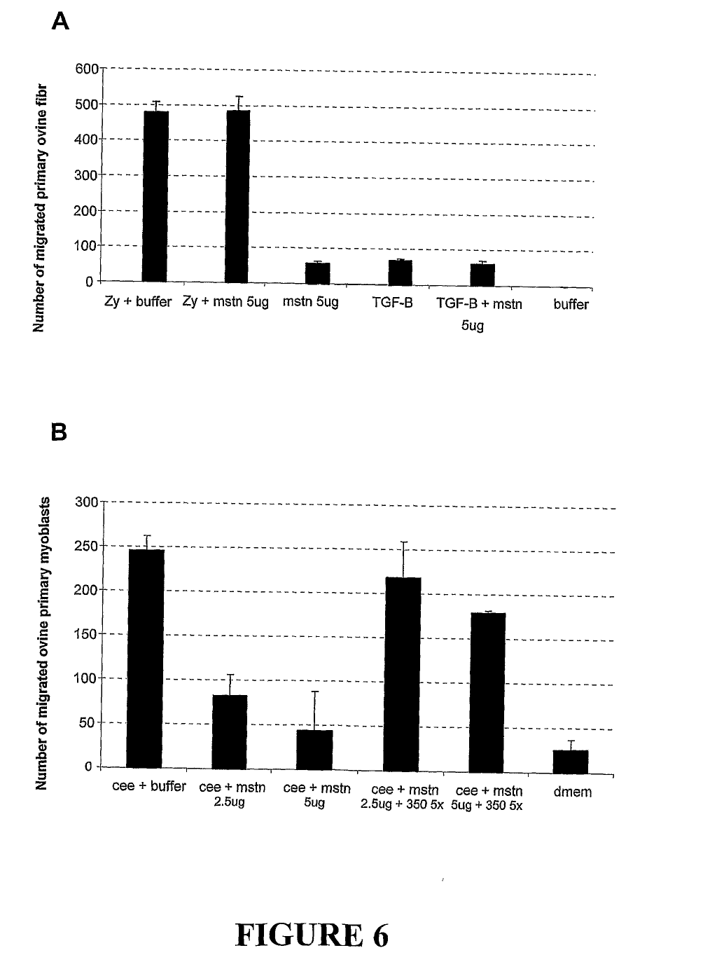

In contrast to the macrophages and myoblasts, myostatin acts as a chemotactic agent for the migration of fibroblasts. This is supported by the observation of reduced migration of fibroblasts to the wound site in the myostatin null muscle (FIG. 6). To directly demonstrate the chemotactic effect of myostatin on the fibroblast, a migration assay was conducted in vitro using recombinant myostatin. As shown in FIG. 6, addition of myostatin increases the chemotactic movement of fibroblasts as compared to the buffer control.

### Example 2

### Antagonizing Myostatin Results in Reduced Fibrosis and Enhanced Muscle Healing

### Methods

### Cut Wound Model

A 3 mm transversal incision was made on the left tibialis anterior (TA) of each mouse (wild type and myostatin null). On days 0, 3, 5, and 7 after wounding the TAs of wild type were injected with either 350 protein at 2 μg/g body weight (total of 85 μg/mouse) or saline at the site of wounding (into the TA muscle). The uninjured right TA was used as control. The injured and control muscle were collected at day 2, 4, 7, 10 and 21 after wounding and their weights determined. The extent of collagen deposition in healing and healed cut wounds was also measured by Van Giesson staining.

### SE Microscopy

The muscle samples were cleaned of fat and tendons and fixed in 10 ml of 0.1 M phosphate buffer (pH 7.4) containing 2.5% (v/v) glutaraldehyde for 48 hours with gentle rocking. The glutaraldehyde was washed off in PBS for 1 hour, before being transferred to 50 mls of 2 M NaOH, and incubated for 5 days at a constant 25° C. Samples were then washed in PBS, and transferred to 50 mls of sterile distilled water. Muscles were kept at a constant 25° C. for an additional 4 days. For the first 36 hours the water was changed every 12 hours, then every 24 hours there after. The muscles were then transferred to 1% tanic acid for 2 hours, and then washed in PBS 3 times. Muscle was treated with 1% OsO4 for 2 hours followed by dehydration by emersion 3 times for 15 min each into an ascending gradient of ethanol (50%-100%). Muscle samples were dried using carbon dioxide and coated with gold. Specimens were examined and photographed using a scanning electron microscope (HITACHI 4100, Japan) with an accelerating voltage of 10 kV.

Collagen accumulation was assessed at day 21 in wild type versus null cut wounded TAs using Geisen as described in Example 1.

### Results

### Lack of Myostatin Results in Enhanced Muscle Healing and Reduced Fibrosis

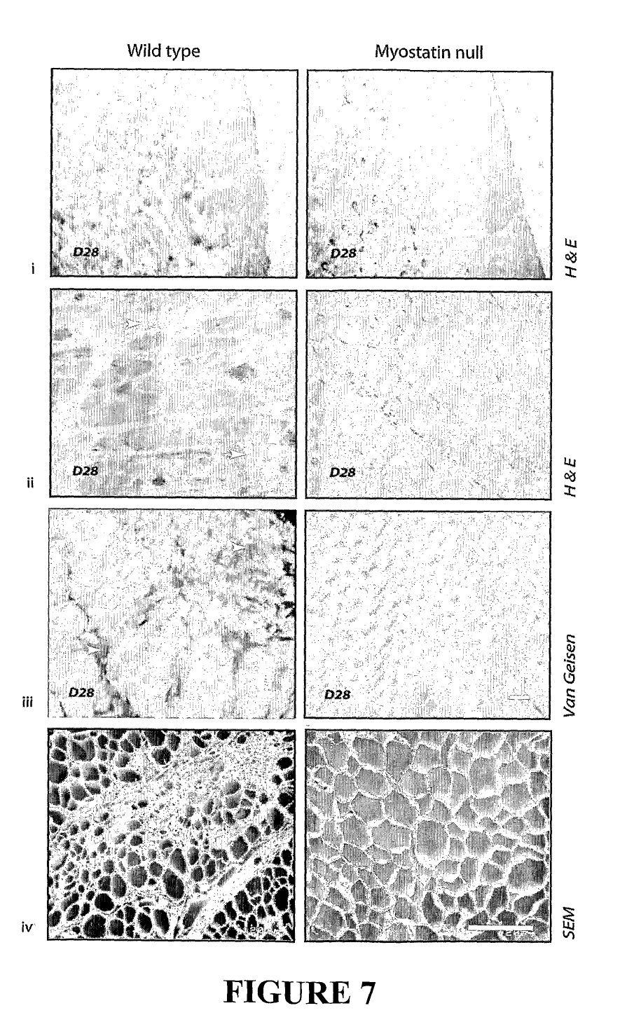

In skeletal muscle, the development of fibrosis begins 2 weeks after notexin wounding and continues over time²⁴. To assess the role of myostatin in fibrosis, histology of both muscle genotypes were compared after notexin wounding (see methods section in Example 1). At day 28, scar tissue was observed in hematoxylin and eosin stained sections from wounded wild type muscle, while very little was seen in the Mstn^{-/-} muscle sections (FIG. 7). The presence of connective tissue was further confirmed by Van Geisen's stain (FIG. 7). Wild type muscle sections at day 28 had larger areas of collagen, therefore more scar tissue was seen in the wounded wild type tissue as compared to the Mstn^{-/-} muscle. To further confirm this result, regenerated muscle was analyzed using scanning electron microscopy. Scanning electron micrographs of day 0 (control) and day 24 regenerated muscle, showed the connective tissue framework surrounding the spaces once occupied by the myofibers (FIG. 7). Neither wild type nor Mstn^{-/-} muscle had thickened connective tissue around the fiber cavity in the control (not injured) samples. However, by day 24 of wound healing dense bundles of connective tissue were observed in the wild type muscle (FIG. 7), but not in the Mstn^{-/-} muscle. Similarly, in a cut wound model comparing myostatin null versus wild type mice the degree of collagen accumulation at the repaired wound site at day 28 was significantly reduced in myostatin null mice (data not presented). These results confirm that lack of myostatin leads to reduced scar tissue after wounding.

### 350 Treatment Enhances Muscle Wound Healing and Reduces Fibrosis

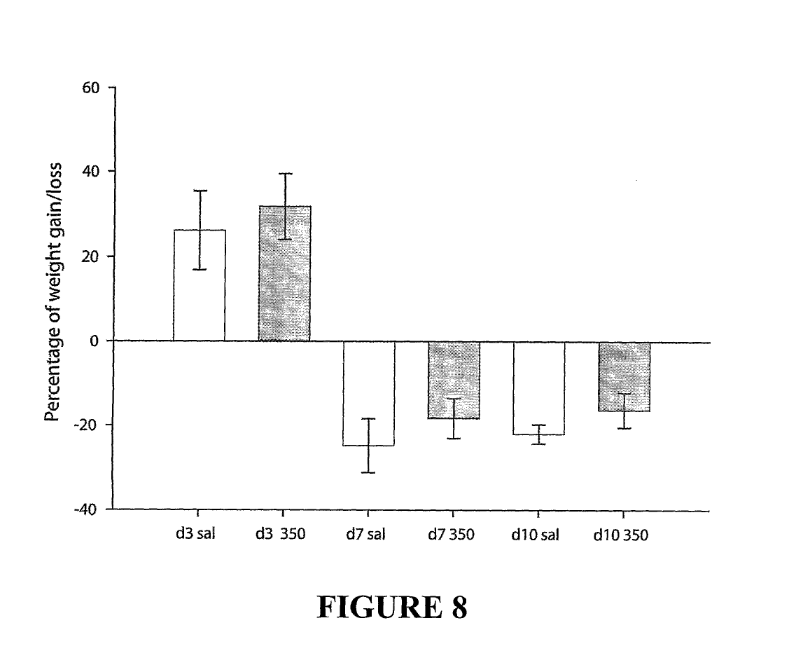

In order to study the efficacy of myostatin antagonists such as 350 in enhancing the wound healing, 1 year old wild type mice (C57 Black) were injured with notexin and injected with 350 (see methods in example 1). After a notexin type wounding, typically the muscle weight initially increases due to the resulting oedema, followed by a decrease due to necrosis of the damaged muscle fibres which are cleared from the site of wounding. After this time, the muscle weight begins to increase again due to growth of new fibres. Results from the trial show that 350 treated muscles do not lose as much weight as control saline injected muscle do (FIG. 8) at day 7 and 10 This is probably due to faster repair of damaged muscle. Molecular data presented (FIG. 4) does indeed support the hypothesis that in 350 treated mice, the damaged muscle healed much faster due to a combination of accelerated and enhanced macrophage migration and the other accelerated wound healing processes discussed earlier that are associated with the use of myostatin antagonists on wound healing.

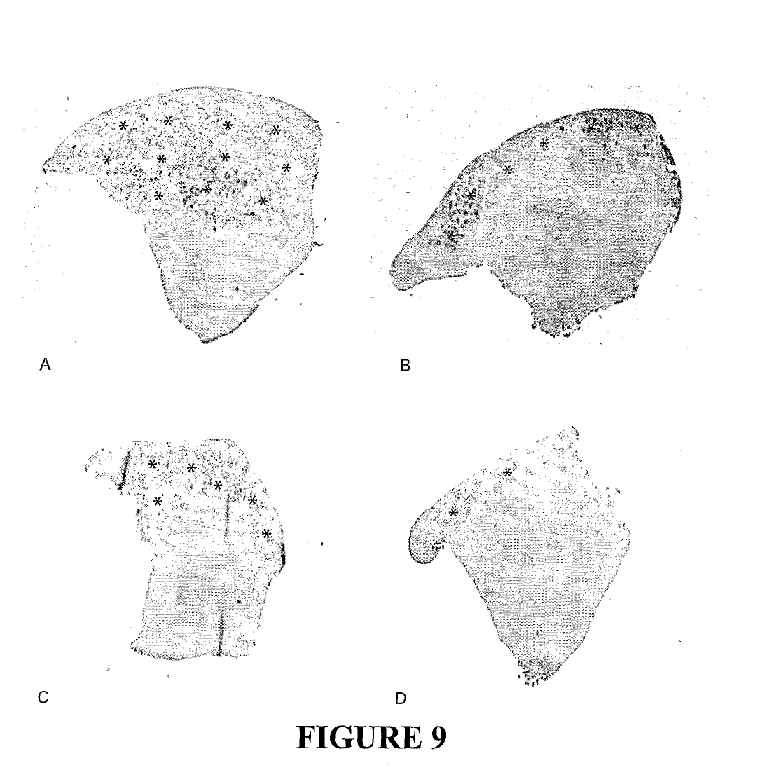

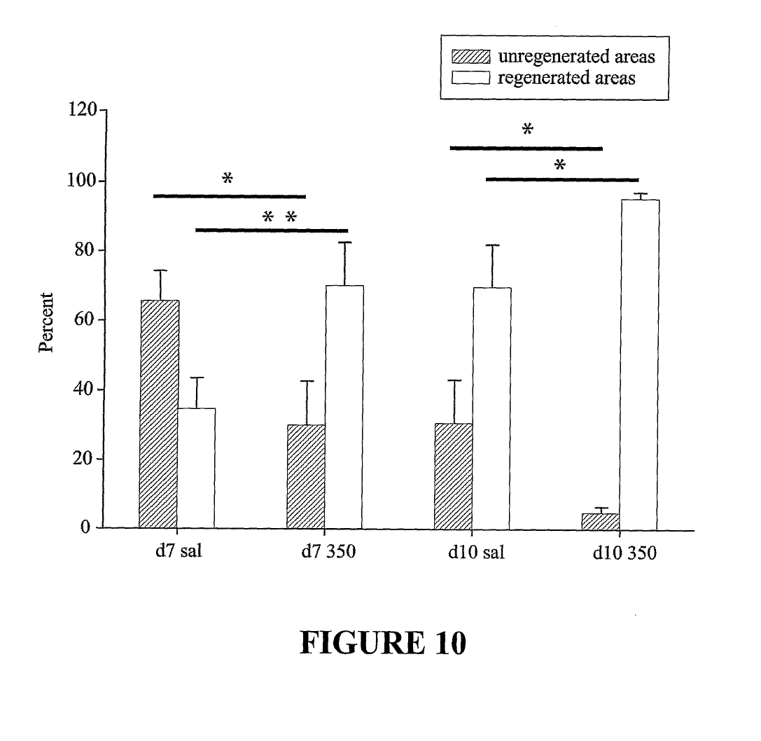

Histological analysis confirmed variations between the saline and 350 treated muscles. Haematoxylin and eosin staining indicated earlier nascent muscle fibre formation and an associated earlier reduction in necrotic areas in the muscles treated with 350 compared to saline treated muscles (FIG. 9). This result confirms accelerated and enhanced muscle wound healing in 350 treated mice. The histological data shown in FIG. 9 was analysed to quantify both healed and non-healed areas of the whole muscle cross-sectional view area. The muscle sections were consistently taken from the mid belly region of each muscle. The analysis shown in FIG. 10, indicates that at day 7 in the saline treated control mice there is increased non-healed area as compared to 350 treated mice. As a result there is a relatively greater muscle tissue loss in controls as compared to 350 treated mice at day 7. The same effect is seen at day 10 also. These results confirm that treatment with the 350 protein results in less muscle tissue loss in muscles recovering from a wound injury. This would be expected to result in improved functionality of the healed muscle.

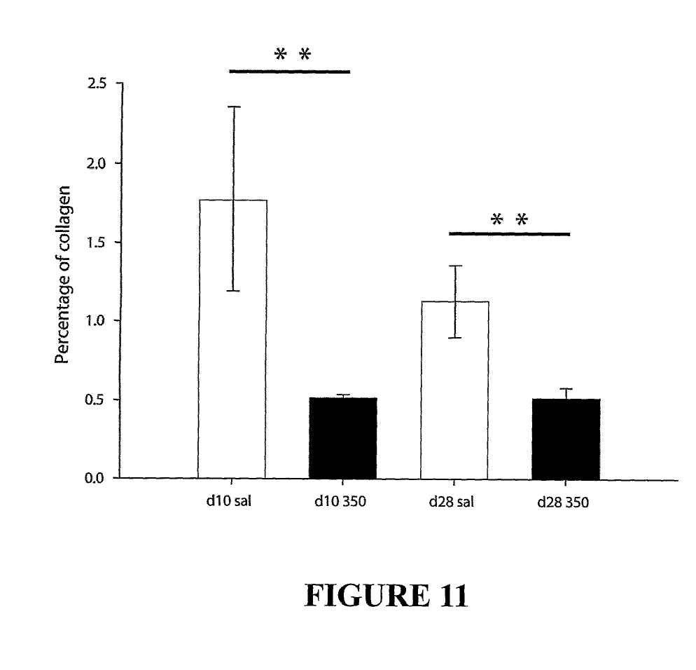

In addition, Van Geisen staining, which detects collagen, showed reduced levels of collagen deposition in 350 treated muscles compared to saline treated muscles, at 10 and 28 days after the administration of notexin indicating that the 350 treatment reduced fibrosis during the wound healing process (FIG. 11). This result demonstrates that myostatin antagonists such as 350 reduce scar tissue (fibrosis) formation during wound healing. Again, less scar tissue and increased muscle tissue would significantly increase the functionality of the healed muscle treated with 350 compared to controls.

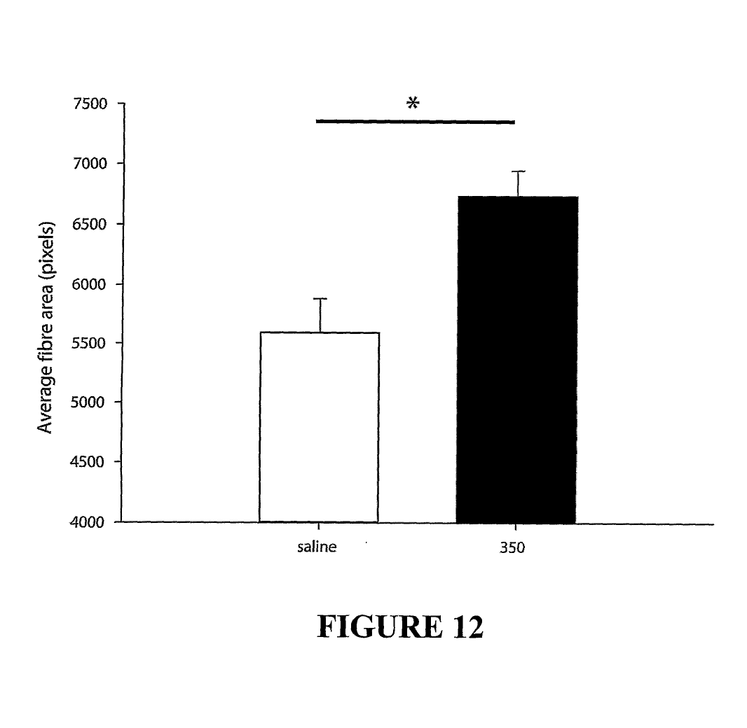

Using the Van Geisen stained images, randomly selected regenerated fibre areas were measured to assess fibre size at 28 days after the administration of notexin (FIG. 12). Results from this analysis indicated that the recovered muscle fibres from 350 treated muscles were significantly larger than the saline treated muscles. The increased repaired muscle fiber size confirms the induction of hypertrophy in muscle cells due to inhibition of myostatin function by 350.

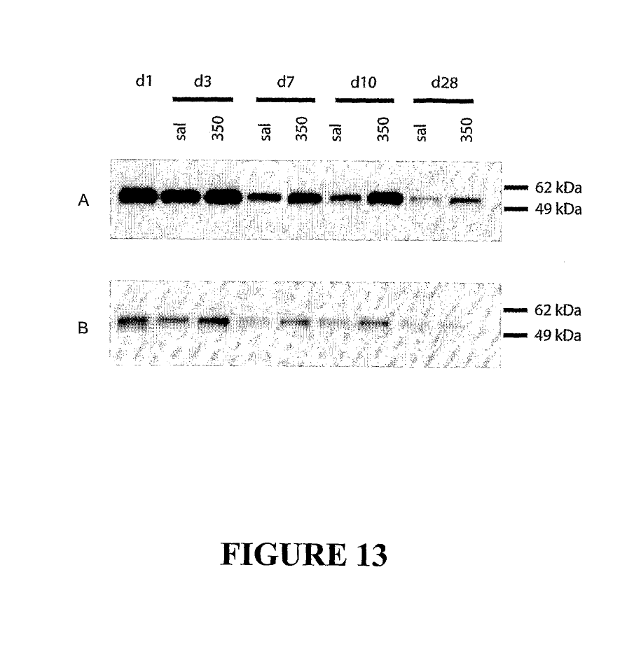

To further confirm that increased wound healing in 350 treated mice is due in part to increased activation of satellite cells we performed molecular analysis for the expression of Pax7 and MyoD proteins. Pax7 protein is a marker for satellite cells and expression of MyoD indicate the activation of satellite cells. Protein analysis confirmed increased levels of satellite cell and activation (FIG. 13). Pax7 levels (FIG. 13A) were higher with 350 treatment at days 3, 7, 10, and 28, indicating an increase in satellite cell activation compared to saline treated muscles. In addition, in the 350 treated muscles, the level of Pax7 increased between day 7 and 10 in contrast to a decrease observed in the saline treated muscle. This would indicate an increase of satellite cell activation around day 10 in the 350 treated muscles. MyoD levels (FIG. 13B) were also higher with 350 treatment at days 3, 7, and 10 showing increased myogenesis compared to the saline treated muscles. Taken together, higher Pax7 and MyoD levels in 350 treated tissues support the observation that activation of satellite cells, and therefore subsequent myogenesis is increased. This result confirms that treatment with 350 accelerates and enhances wound healing.

### Local Application of 350 Induced Enhanced Wound Healing.

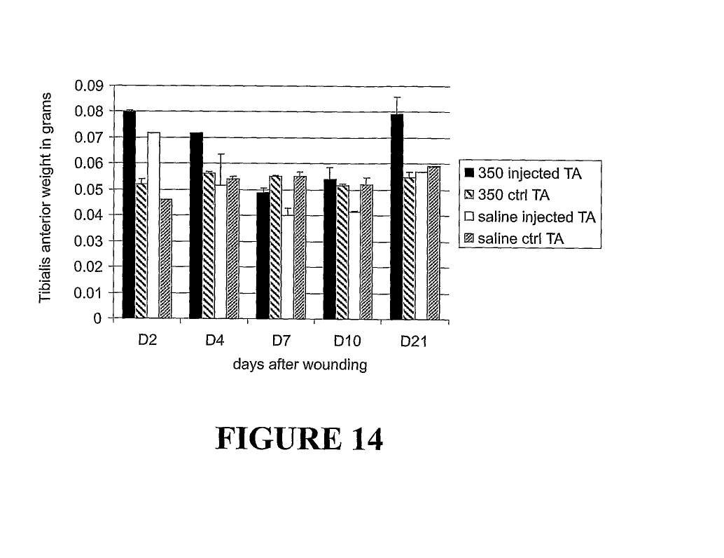

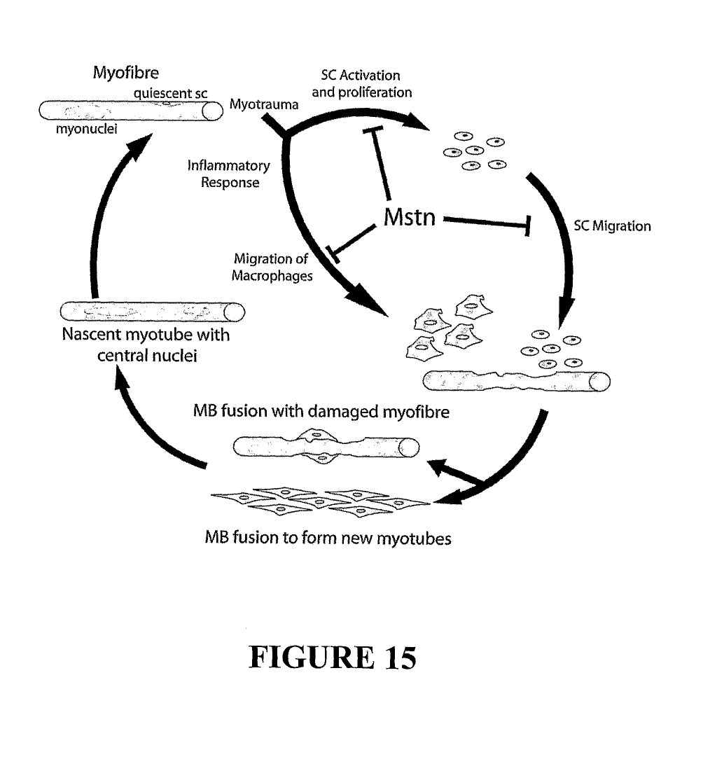

To assess the effectiveness of direct application of 350 at the wound site in enhancing wound healing, 350 protein was applied to the TA muscle that was regenerating after cut wounding. The uninjured right TA was used as control. The injured and control muscles were collected at day 2, 4, 7, 10 and 21 after wounding and their weight determined. An initial increase in muscle weight due to inflammatory infiltration is observed in both 350 and saline injected TAs at day 2 and 4 after wounding (FIG. 14). At day 7 to 10 after wounding the muscles recover their normal weight in both 350 and saline injected TAs. However, at day 21 after wounding, the 350 injected TAs display a significant increase in muscle size as reflected in muscle weight compared to saline treated muscles.

### DISCUSSION

Myostatin is a potent negative regulator of myogenesis. Surprisingly, the current results demonstrate that myostatin is also involved in regulating inflammatory response and there by controls the muscle healing process and scar tissue formation. As part of the normal wound healing process macrophages infiltrate the wound site soon after wounding and by release of chemokines contribute to key processes in healing such as regulation of epitheliasation, tissue remodeling and angiogenesis in skin²⁵ and other tissues.

Histological data clearly demonstrates that there is increased and accelerated infiltration of macrophages and myoblasts into the wound area of the tibialis anterior muscle of Mstn^{-/-} mice, compared to the wild type mice (FIG. 1). Secondly, in the Mstn^{-/-} mice, a majority of the muscle fibers lost are replaced by new muscle fibers while accumulation of connective tissue is reduced (FIG. 7). In injured muscle, the damaged myofibers undergo necrosis. During wound healing the necrotic area is invaded by small blood vessels, mononuclear cells and activated macrophages. These activated lymphocytes simultaneously secrete several cytokines and growth factors, which are critical in chemotaxis and subsequent wound healing processes. More importantly, the release of growth factors at the injured site also regulates myoblast migration, proliferation and differentiation to promote muscle wound healing and repair²⁶. Myostatin antagonists have been shown here to increase tissue repair by increased earlier accumulation of myogenic cells leading to accelerated healing.

It has been shown previously that myostatin is present in the wound site soon after wounding in a number of wound types. It has been shown here that myostatin inhibits migration of macrophages and myoblasts in chemotaxis experiments. Importantly, addition of myostatin antagonists such as 350 successfully overcomes the negative effects of myostatin on migration of both myoblasts and macrophages. Thus when injured tissues are treated with myostatin antagonists, accelerated and enhanced migration of macrophages and myoblasts to the wound site results in improved wound healing. Our results show that the potent myostatin antagonist 350 when injected into mice undergoing wound healing results in improved wound healing.

Fibrosis is a part of the wound healing processes but excess fibrosis leads to scarring and reduced function of tissues. Fibroblasts play a major role in deposition of collagen and thus scar formation in wounds. Studies have previously correlated the extent of fibroblast accumulation with scarring in skin burn wounds¹⁴. We have shown here that myostatin is a potent chemo-attractant of fibroblasts and it has been shown previously that myostatin accumulates at increased levels in wounded tissues soon after wounding. In myostatin null mice there is decreased accumulation of fibroblasts at a cut wound site and a consequent decrease in scarring in the healed wound. Importantly, the data presented here shows that the capacity to antagonize myostatin by local and systemic administration of antagonists consequently leads to decreased collagen accumulation and scarring in tissue that has undergone wound healing. Collagen has been found to be the major pathological finding in a number of fibrotic diseases²⁷. It is therefore expected that other medical conditions such as cystic fibrosis, fibrocystic disease of the pancreas, mucoviscidosis, pancreatic fibrosis, myelofibrosis, idiopathic pulmonary fibrosis, hepatic fibrosis, scleroderma, osteogenesisimperfecta or any other fibrotic conditions that are characterised by excessive deposition of collagen and fibrotic tissue can be treated by administration of myostatin inhibitors.

### CONCLUSION

Myostatin inhibitors, applied systemically and locally, have been shown here to increase the rate of wound healing by acceleration and enhancement of several key processes. The application of myostatin inhibitors has also been shown to result in decreased deposition of collagen at the final healed wound site which prevents loss of tissue function or cosmetic damage due to scarring.

It is not the intention to limit the scope of the invention to the abovementioned examples only. As would be appreciated by a skilled person in the art, many variations are possible without departing from the scope of the invention (as set out in the accompanying claims).

### REFERENCES

1. Sedlarik K. M; The Process of Wound Healing. Wung Forum. Published on-line: hartmann_online, 1994
2. Laurence Rosenberg; Wound Healing, Growth Factors. Published on-line: eMedicine. 15 May 2003
3. Michael Mercandetti; Wound Healing, Healing and Repair. Published on-line: eMedicine. 1 Aug. 2005
4. Anderson, J. E. (1998). Murray L. Barr Award Lecture. Studies of the dynamics of skeletal muscle regeneration: the mouse came back! Biochem Cel Biol 76, 13-26.
5. Poor/Slow Wound Healing. On-line publication: Diagnose-me.com. 22 Nov. 2005.
6. McPherron Ac, Lee S J (1997). Double Muscling in cattle due to mutations in the myostatin gene PNAs 19: 12457-12461.
7. McPherron A C, Lawler A M and Lee S J (1997). Regulation of skeletal muscle mass in mice by a new TGF-beta super family member. Nature 387, 83-90.
8. Lee S J and McPherron A C (2001). Regulation of myostatin activity and muscle Growth. Procedings of National Academy of Science 98, 9306-9311
9. Rayburn E., Wang w., Zhang R and Wang H. (2005) Antisense approaches in drug discovery and development. Progress in Drug Research 63: 227-74.
10. Ren Y., Gong W., Xu Q., Zheng X., Lin D., Wang Y. and Li T. (2006) siRecords: an extensive database of mammalian siRNAs with efficacy ratings. Bioinformatics 29:epublished
11. Nakamura K., Murata C., Ito M., Iwamori T., Nishimura S., Hisamatsu K., Sonoki S., Nakayama A., Suyama E., Kawasaki H., Taira k., Nishino K. and Tachi C. (2005) Design of hammerheas ribozymes that cleave murin sry mRNA in vitro and in vivo. Journal of Reproductive Development 17: epublished.
12. Kirk S., Oldham J., Kambadue R., Sharma., Dobbie P. and Bass J. (2000). Myostatin regulation during skeletal muscle regeneration. Journal of Cellular Physiology 184(3): 356-63.
13. Gillitzer R. and Goebeler M. (2001). Chemokines in cutaneous wound healing. Journal of Leukocyte Biology 69(4): 513-21.
14. Yang L., Scott P. G., Dodd C., Medina A., Jiao H., Shankowsky H. A., Ghahary A. and Tredget E. E. (2005). Identification of fibrocytes in postburn hypertrophic scar. Wound Repair and Regeneration 13(4): 398-404.
15. Springer, T., Galfre, G., Secher, D. S. and Milstein, C. (1979). Mac-1: a macrophage differentiation antigen identified by monoclonal antibody. Eur J Immunol 9, 301-6.
16. Allen, R. E., Temm-Grove, C. J., Sheehan, S. M. and Rice, G. (1997). Skeletal muscle satellite cell cultures. Methods Cel Biol 52, 155-76.
17. Patridge, T. A. (1997). Tissue culture of skeletal muscle. Methods Mol Biol 75, 131-44.
18. Watt, D. J., Morgan, J. E., Clifford, M. A. and Patridge, T. A. (1987). The movement of muscle precursor cells between adjacent regenerating muscles in the mouse. Anat Embryol (Berl) 175, 527-36.
19. Beauchamp, J. R., Heslop, L., Yu, D. S., Tajbakhsh, S., Kelly, R. G., Wernig, A., Buckingham, M. E., Patridge, T. A. and Zammit, P. S. (2000). Expression of CD34 and Myf5 defines the majority of quiescent adult skeletal muscle satellite cells. J Cel Biol 151, 1221-34.
20. Kawakami, K., Teruya, K., Tohyama, M., Kudeken, N., Yonamine, Y. and Saito, A. (1995). Mac1 discriminates unusual CD4−CD8− double negative T cells bearing alpha beta antigen receptor from conventional ones with either CD4 or CD8 in murine lung. ImImmunol Lett 46, 143-52.
21. Grounds, M. D., Garrett, K. L. and Beilharz, M. W. (1992). The transcription of MyoD1 and myogenin genes in thymic cells in vivo. Exp. Cel Res 198, 357-61.
22. Bischoff, R. (1997). Chemotaxis of skeletal muscle satellite cells. Dev Dyn 208, 505-15. Bischoff, R. and Heintz, C. (1994). Enhancement of skeletal muscle regeneration Dev Dyn 201, 41-54.
23. Jones, G. E. (2000). Cellular signaling in macrophage migration and chemotaxis. J Leukoc Biol 68, 593-602.
24. Sato, K., Li, Y., Foster, W., Fukushima, K., Badlani, N., Adachi, N., Usas, A., Fu, F. H. and Huard, J. (2003). Improvement of muscle healing through enhancement of muscle regeneration and prevention of fibrosis. Muscle Nerve 28, 365-72.
25. Gillitzer R and Goebeler M, (2001). Chemokines in cutaneous wound healing. Journal of Leukocyte Biology. 69, 513-21
26. Moens, P. D., Van-Schoor, M. C. and Marechal, G. (1996). Lack of myoblasts migration between transplanted and host muscles of mdx and normal mice. J. Muscle Res Cel Motil 17, 37-43.
27. Bhogal R K, Stoica C M, McGaha T L and Bona C A. Molecular aspects of regulation of collagen gene expression in fibrosis. (2005) Journal of Clinical Immunology 25(6) 592-603.

### Cited Patent Documents

U.S. Pat. No. 6,096,506, U.S. Pat. No. 6,468,535, U.S. Pat. No. 6,369,201, US2004/0181033, WO 01/05820, WO 02/085306, WO 00/43781, WO 01/53350, PCT/NZ2005/000250, and PCT/NZ2004/000308.

All of the references and cited patent documents are hereby incorporated into the present specification by reference.

### INDUSTRIAL APPLICATION

The present invention provides a method for improving wound healing by administering either systemically or locally one or more myostatin antagonists. The method provides for improved wound healing time, as well as a reduction in scar tissue formation and reduced loss of tissue function. The method will be particularly useful in cosmetic treatments.

1. A method of improving tissue wound healing comprising the step of administering an effective amount of at least one myostatin antagonist to a human or non-human patient in need thereof.

2. A method as claimed in claim 1, wherein the at least one myostatin antagonist is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

3. A method as claimed in claim 2, wherein the at least one myostatin antagonist is a dominant negative of myostatin selected from the Piedmontese allele and mature myostatin peptides having a C-terminal truncation at a position at of between amino acid positions 335 to 375.

4. A method as claimed in claim 3, wherein the at least one myostatin antagonist is a mature myostatin peptide having a C-terminal truncation at amino acid position 335 or 350.

5. A method as claimed in claim 2, where the at least one myostatin antagonist is a splice variant of myostatin selected from a polypeptide of SEQ ID NOS: 8-14, or a functional fragment or variant thereof, or a sequence having 95%, 90%, 85%, 80%, 75% or 70% sequence identify thereto.

6. A method as claimed in claim 2, wherein the at least one myostatin antagonist is a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

7. A method as claimed in claim 1, for improving healing of a superficial skin wound, including cuts and abrasions; deep wound extending through the skin and muscle, including surgical incisions; internal wounds, including wounds to muscle and tendon caused by sports injury or trauma, bruises and hematomas; and burns.

8. A method as claimed in claim 1, wherein one or more additional immuno-responsive compounds selected from the group consisting of glucocorticosteroids, non-steroidal anti-inflammatory drugs (NSAIDs), PDGF, EGF, IGF, and TNF-alpha antagonists are co-administered either separately, sequentially or simultaneously with the at least one myostatin antagonist to further improve wound healing.

9. A method as claimed in claim 1, wherein the at least one myostatin antagonists is formulated for local or systemic administration.

10. A method as claimed in claim 9, wherein the at least one myostatin antagonist is formulated for oral, intravenous, cutaneous, subcutaneous, intradermal, topical, nasal, pulmonary, intramuscular or intraperitional administration.

11. A use of at least one myostatin antagonist in the manufacture of a medicament for improving tissue wound healing in a human or non-human patient in need thereof.

12. A use as claimed in claim 11, wherein the at least one myostatin antagonist is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

13. A use as claimed in claim 12, wherein the at least one myostatin antagonist is a dominant negative of myostatin selected from the Piedmontese allele and mature myostatin peptides having a C-terminal truncation at a position at of between amino acid positions 335 to 375.

14. A use as claimed in claim 13, wherein the at least one myostatin antagonist is a mature myostatin peptide having a C-terminal truncation at amino acid position 335 or 350.

15. A use as claimed in claim 12, where the at least one myostatin antagonist is a splice variant of myostatin selected from a polypeptide of SEQ ID NOS: 8-14, or a functional fragment or variant thereof, or a sequence having 95%, 90%, 85%, 80%, 75% or 70% sequence identify thereto.

16. A use as claimed in claim 12, wherein the at least one myostatin antagonist is a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

17. A use as claimed in claim 11, for improving healing of a superficial skin wound, including cuts and abrasions; deep wound extending through the skin and muscle, including surgical incisions; internal wounds, including wounds to muscle and tendon caused by sports injury or trauma, bruises and hematomas; and burns.

18. A use as claimed in claim 11, wherein the medicament further comprises one or more additional immuno-responsive compounds selected from the group consisting of glucocorticosteroids, non-steroidal anti-inflammatory drugs (NSAIDs), PDGF, EGF, IGF, and TNF-alpha antagonists, and wherein the medicament is formulated for separate, sequential or simultaneous administration of the at least one myostatin antagonist and additional compound.

19. A use as claimed in claim 11, wherein the medicament is formulated for local or systemic administration.

20. A use as claimed in claim 19, wherein the medicament is formulated for oral, intravenous, cutaneous, subcutaneous, intradermal, topical, nasal, pulmonary, intramuscular or intraperitional administration.

21. A pharmaceutical compound comprising at least one myostatin antagonist and a pharmaceutically acceptable carrier, when used in a method of improving wound healing in a human or non-human patient in need thereof.

22. A pharmaceutical compound as claimed in claim 21, wherein the at least one myostatin antagonist is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

23. A pharmaceutical compound as claimed in claim 22, wherein the at least one myostatin antagonist is a dominant negative of myostatin selected from the Piedmontese allele and mature myostatin peptides having a C-terminal truncation at a position at of between amino acid positions 335 to 375.

24. A pharmaceutical compound as claimed in claim 23, wherein the at least one myostatin antagonist is a mature myostatin peptide having a C-terminal truncation at amino acid position 335 or 350.

25. A pharmaceutical compound as claimed in claim 22, where the at least one myostatin antagonist is a splice variant of myostatin selected from a polypeptide of SEQ ID NOS: 8-14, or a functional fragment or variant thereof, or a sequence having 95%, 90%, 85%, 80%, 75% or 70% sequence identify thereto.

26. A pharmaceutical compound as claimed in claim 22, wherein the at least one myostatin antagonist is a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

27. A pharmaceutical compound as claimed in claim 21, for improving healing of a superficial skin wound, including cuts and abrasions; deep wound extending through the skin and muscle, including surgical incisions; internal wounds, including wounds to muscle and tendon caused by sports injury or trauma, bruises and hematomas; and burns.

28. A pharmaceutical compound as claimed in claim 21, further comprising one or more additional immuno-responsive compounds selected from the group consisting of glucocorticosteroids, non-steroidal anti-inflammatory drugs (NSAIDs), PDGF, EGF, IGF, and TNF-alpha antagonists, wherein the composition is formulated for separate, sequential or simultaneous administration with the at least one myostatin antagonist.

29. A pharmaceutical composition as claimed in claim 21, formulated for local or systemic administration.

30. A pharmaceutical compound as claimed in claim 29, formulated for oral, intravenous, cutaneous, subcutaneous, intradermal, topical, nasal, pulmonary, intramuscular or intraperitional administration.

31. At least one myostatin antagonist when used in a method of improving wound healing in a human or non-human patient in need thereof.

32. At least one myostatin antagonist as claimed in claim 31, is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

33. At least one myostatin antagonist as claimed in claim 32, comprising a dominant negative of myostatin selected from the Piedmontese allele and mature myostatin peptides having a C-terminal truncation at a position at of between amino acid positions 335 to 375.

34. At least one myostatin antagonist as claimed in claim 33, comprising a mature myostatin peptide having a C-terminal truncation at amino acid position 335 or 350.

35. At least one myostatin antagonist as claimed in claim 32, comprising a splice variant of myostatin selected from a polypeptide of SEQ ID NOS: 8-14, or a functional fragment or variant thereof, or a sequence having 95%, 90%, 85%, 80%, 75% or 70% sequence identify thereto.

36. At least one myostatin antagonist as claimed in claim 32 comprising a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

37. At least one myostatin antagonist as claimed in claim 31 for improving healing of a superficial skin wound, including cuts and abrasions; deep wound extending through the skin and muscle, including surgical incisions; internal wounds, including wounds to muscle and tendon caused by sports injury or trauma, bruises and hematomas; and burns.

38. At least one myostatin antagonist as claimed in claim 31 in combination with one or more additional immuno-responsive compounds selected from the group consisting of glucocorticosteroids, non-steroidal anti-inflammatory drugs (NSAIDs), PDGF, EGF, IGF, and TNF-alpha antagonists for separate, sequential or simultaneous administration with the at least one myostatin antagonist to further improve wound healing.

39. At least one myostatin antagonists as claimed in claim 31, formulated for local or systemic administration.

40. At least one myostatin antagonist as claimed in claim 39 formulated for oral, intravenous, cutaneous, subcutaneous, intradermal, topical, nasal, pulmonary, intramuscular or intraperitional administration.

41. A method of treating fibrotic diseases or disorders comprising administering to a patient in need thereof a therapeutically effective amount of a myostatin antagonist.
